(19) United States
(12) Patent Application Publication
(10) Pub. No.: US 2005/0033352 A1
(43) Pub. Date: Feb. 10, 2005

(54) Actuating drive for surgical instruments

(76) Inventors: Christoph Zepf, Durbheim (DE); Helmut Kratt, Spaichingen (DE); Ilyas Guel, Villingen-Schwenningen (DE)

Correspondence Address: MCGLEW & TUTTLE, PC, 1 SCARBOROUGH STATION PLAZA, SCARBOROUGH, NY, 10510-0827, US

(21) Appl. No.: 10/877,147

(22) Filed: Jun. 25, 2004

(30) Foreign Application Priority Data
Jun. 27, 2003 (DE) DE 103 28 934.8

(51) Int. Cl. A61B 17/24

(52) U.S. Cl. 606/205

(57) ABSTRACT

A surgical instrument (1) with a grip part (2), which is provided with a working part (26), which has a tool part in an end area and is provided with a slide (57). The slide (57) is adjustable in relation to the working part (26). The adjusting movement (double arrow 18) brings about a working movement of the tool part. The adjusting movement (double arrow 18) can be brought about by an actuating drive (6, 7, 8, 9, 10), which can be activated by an actuating element (38), which can be actuated manually. To attain the strongest possible actuating forces, a control means (35) is provided, which can be controlled via the actuating element (38) and by which the actuating drive (6, 7, 8, 9, 10) arranged in the area of the grip part (2) can be actuated. The actuating drive (6, 7, 8, 9, 10) now brings about the working movement of the slide (57) depending on the position of the actuating element (38) and is mechanically coupled with the slide (57) via a lever mechanism (10).

### CROSS REFERENCE TO RELATED APPLICATIONS

This application claims the benefit of priority under 35 U.S.C. § 119 of DE 103 28 934.8 filed Jun. 27, 2003, the entire contents of which are incorporated herein by reference.

### FIELD OF THE INVENTION

The present invention pertains to a surgical instrument with a grip part, which is provided with a working part, which has a tool part in an end area and is provided with a slide, which is adjustable in relation to the working part and whose adjusting movement brings about a working movement of the tool part, wherein the adjusting movement can be brought about by an actuating drive, which can be actuated via an actuating element, which can be actuated manually.

### BACKGROUND OF THE INVENTION

Many different embodiments of surgical instruments of this type have been known. Reference is made in this connection to DE 93 07 793 U1, DE 29 71 4735 U1 as well as DE 93 17 488 U1. Further proof of the state of the art of such surgical instruments of this type can be found in these documents. Furthermore, such surgical instruments have also become known from DE 29 71 8969 with further proof

The common feature of these surgical instruments is that they have a working part, which is provided with a tool part. The tool parts may have different shapes and have different functions. For example, forceps-like tool parts are known, with which it is possible, for example, to take tissue samples. In addition, scissor-like tool parts are also known, which can be used in a similar manner to remove tissue samples or also to sever adhesions. So-called punching tools of so-called bone punches, which are used to punch out or remove projecting bone or cartilage parts, are known as other tool parts as well.

Furthermore, it is common to these surgical instruments that they have a slide adjustable in relation to the working part in their working part or at their working part. To drive the particular tool part intended, this slide is displaceable along the working part, so that the tool part performs a corresponding working movement. This working movement of the particular tool part is brought about, as a rule, by the manual actuation of an actuating element. As scissor-like gripping elements, such actuating elements may form a kind of grip part or they may also be arranged as a pivotable hand lever at a grip part.

It was now found, especially where stronger forces and/or also a higher precision are necessary during the use of a surgical instrument, that the manual actuation of these working parts always leads to unintended deflections of the tool tip with its tool part. Furthermore, rapid fatiguing of the user can be observed in case of a high repetition rate and/or when a strong force must be applied to the grip part, which is highly disadvantageous especially in case of prolonged surgical procedures.

A surgical instrument in which the working movement of the push rod and consequently of the tool is brought about by a drive is known from DE 41 06 797 A1. The cutting force is said to be automatically controlled and the user of the instrument to be relieved thereby. The drive comprises in this case an electric motor, which rotatingly drives a drive shaft via a gear mechanism. The drive shaft is provided at its free end with a pinion, which meshes with a toothed rack profile of the push rod. The rotary movement of the drive shaft is converted by this rack and pinion gear into an axial movement of the push rod. It was now found that such a rack and pinion gear is highly error-prone for strong shear forces, especially during longer operating times, and that very strong shear forces cannot be attained, because this entails a great wear of the teeth and consequently the destruction of the rack and pinion gear.

### SUMMARY OF THE INVENTION

The basic object of the present invention is correspondingly to improve a surgical instrument of this type such that strong shear forces can be brought about to bring about the adjusting movement of the push rod even during longer operating times.

The object is accomplished according to the present invention with a surgical instrument having a grip part and a working part having a tool part in an end area provided with a slide, which is adjustable in relation to said working part and has an adjusting movement that brings about a working movement of said tool part and with an actuating element which can be actuated manually and an actuating drive for bringing about said adjusting movement. A control means can be controlled via the actuating element and actuates the actuating drive arranged in the area of the grip part. The actuating drive brings about the working movement of the slide depending on the position of the actuating element and is mechanically coupled with the slide via a lever mechanism.

The design according to the present invention provides a surgical instrument that can be operated in a very simple manner and with which strong working forces can be applied to the tool part at high repetition rates. It is ensured that the operator can control the surgical instrument according to the present invention during the use precisely at the work site. The operator experiences hardly any fatigue even after prolonged use, so that the surgical instrument according to the present invention with its actuating drive is especially suitable for extensive and time-consuming surgical procedures. Provisions are made for the actuating drive to be coupled with the slide via a lever mechanism. A compact arrangement of the actuating drive within the grip part is possible due to this design. Furthermore, extremely strong driving forces are available due to the lever action of the lever mechanism, which always brings about extremely strong working forces especially when the motor drive is used in a surgical instrument in the form of a so-called bone punch as a tool part, and this instrument can thus be used reliably.

Provisions may be made for the lever mechanism to have a support lever and a pushing lever, and for the support lever and the pushing lever to be connected with one another rotatably via an intermediate joint, and for the support lever to be mounted rotatably with its end located opposite the intermediate joint in the grip part, and for the pushing lever to be connected with its end located opposite the intermediate joint with the slide in an articulated manner via a prismatic joint, and for the support lever and the pushing lever to be able to be brought by the actuating drive for driving the slide from an angular position into an at least approximately aligned position. Due to the lever mechanism being designed according to the present invention as a toggle joint or toggle lever, an extremely simple design is obtained, on the one hand, and strong moving forces can be applied, on the other hand.

Provisions may be made for the actuating drive to have an electric motor, via which a push rod mechanically coupled with the lever mechanism can be driven. The actuating drive can be actuated in a very simple manner due to this design according to the present invention. The manufacture is extremely inexpensive as well. The push rod may be, for example, a threaded spindle, which is in gear connection with the intermediate joint, so that the intermediate joint is deflected by the electric motor during the rotating drive of the threaded spindle, and the support lever and the pushing lever can be brought from their angular starting position into an at least approximately aligned end position.

To prevent the electric motor from being overloaded, provisions may be made for the electric motor to be in gear connection with the push rod via a planet gear. It shall be noted here that the push rod may also be able to be deflected by a cam, and this deflecting movement is transmitted to the lever mechanism. Such a construction is also extremely compact, and strong forces can be transmitted to the slide and consequently to the tool part.

Provisions may be made for the electric motor to be able to be actuated by the control means such that the tool part can be adjusted from a starting position into an end position and vice versa. This means that the electric motor moves the tool part during the actuation of the actuating element from the starting position into a usually predetermined end position. This end position may be, for example, the closed or at least approximately closed gripped position in the case of a forceps-shaped tool part. If the tool part consists of a kind of scissors, they are brought from the opened starting position into the closed end position during the activation of the electric motor in a continuous working movement. Such an actuation can be embodied in an extremely simple manner by the control means, because ultimately only a limit stop is necessary for both directions of movement, and the electric motor only needs to be electrically reversed by the control means for these two directions of movement.

Provisions may be made for the electric motor to be designed as a motor operator, and for the adjustment path of the actuating element to be able to be detected by means of a measuring means and transmitted to the control means, and for the motor operator to be able to be actuated by the control means such that the working movement of the working part is essentially proportional to the adjustment path of the actuating element. Extremely precise handling of the surgical instrument according to the present invention is ensured by this design. Due to the proportional movement of the working part for driving the tool part, be it in the form of a scissors, a gripping forceps or even a punching tool, the necessary activities can be performed by the operator in the area of the surgery just as with a conventional surgical instrument of any type as far as the movements are concerned.

For example, the closing angle can be set precisely for the particular part to be gripped during the closing movement of a forceps-like tool part, on the one hand, and extremely strong forces can also be applied, on the other hand, in order to hold that part in an absolutely firmly seated manner if necessary with the forceps-like tool part, on the other hand. Since the actuating drive with its gear mechanism and its actuation follows the movement of the actuating element proportionally, the surgical instrument according to the present invention can thus be operated in the conventional manner because of the design, so that the operator does not require a phase of getting familiar with the use of this instrument.

Conventional electronic measuring means may be provided as the measuring means, according to which the measuring means is formed by an incremental transducer, especially an incremental shaft encoder.

As another variant, the actuating drive may also be designed as a double-acting or single-acting pneumatic cylinder arranged integrated in the grip part. A pneumatic control valve, which can be actuated via the actuating element, is provided for this type of drive for actuating this pneumatic drive or the adjusting movement of this pneumatic drive. Such a pneumatic cylinder with a corresponding actuation may be provided, for example, for a tool part in the form of a bone punch or also in the form of a forceps. The use of a pneumatic cylinder has the advantage that no proportional control needs to be provided to operate especially a bone punch or a forceps. Especially in case of use for a forceps-like tool part, the opening angle of the tool part automatically adjusts itself during the closing movement to the size of the part to be gripped, and sufficiently strong gripping forces can be applied by the pneumatic cylinder at any desired opening angle of the forceps-like tool part in conjunction with the lever mechanism. In addition, overloading of the pneumatic cylinder is not possible, so that such an actuating drive is extremely robust and has a long service life. An actuating drive in the form of a pneumatic cylinder can be used at low cost wherever proportional control is not necessary for controlling the movement process of the tool part.

To make it possible to use the surgical instrument according to the present invention with its actuating drive as variably and at as low a cost as possible, provisions may be made for the working part with its tool part to be arranged detachably at the grip part. This means in turn that different working parts with different tool parts can be replaced with one another on the grip part in a simple manner, so that only one grip part with an actuating drive is necessary for different intended uses. Extremely inexpensive manufacture is achieved as a result. Furthermore, such a design also offers advantages in terms of hygiene, because only the different working parts with their particular tool parts need to be correspondingly sterilized, while the grip part itself does not need to be subjected to such hygienic measures.

Provisions may be made for the replaceable connection of the working part with the grip part where, the grip part has a housing, which is provided with a coupling housing for coupling with the working part. Due to this two-part design of the housing with its coupling housing, it is possible to make, in particular, the coupling to the working part variable depending on the needs.

Provisions may be made in this connection for the coupling housing to have a closeable receiving chamber, into which the working part with its guide shaft can be replaceably inserted, and for the slide to protrude axially into the receiving chamber from the guide shaft, and for the prismatic joint of the lever mechanism to be provided with a ram, which protrudes into the prismatic joint and with which the slide can be engaged in a positive-locking and detachable manner. This design guarantees an extremely simple replaceability of the different working parts in a simple manner. Thus, the different working parts can be plugged or pushed into the receiving chamber in a simple manner by simply “hanging” them in, and the slide can at the same time be caused to engage the ram of the prismatic joint in a positive-locking manner.

Furthermore, provisions may be made for the receiving chamber of the coupling housing to be able to be closed with a housing cover, which is mounted pivotably on the coupling housing, and for the working part to be held by the closed housing part in the receiving chamber. Replacement of the different working parts can be carried out extremely rapidly and simply due to this design. The housing cover may be held in its closed state, for example, by a locking connection or the like.

Provisions may also be made, as was already stated above, that different tool parts are provided for different intended uses, which different tool parts can be mounted replaceably on the grip part. An extremely variable use of the surgical instrument with its actuating drive is possible due to this design.

An exemplary embodiment of a surgical instrument according to the present invention with an actuating drive will be explained in greater detail below on the basis of the drawings. The various features of novelty which characterize the invention are pointed out with particularity in the claims annexed to and forming a part of this disclosure. For a better understanding of the invention, its operating advantages and specific objects attained by its uses, reference is made to the accompanying drawings and descriptive matter in which preferred embodiments of the invention are illustrated.

### BRIEF DESCRIPTION OF THE DRAWINGS

FIG. 1 is a partial sectional view of the grip part of a surgical instrument according to the present invention;

FIG. 2 is a partial sectional view of the lower end area of the grip part with a flanged battery;

FIG. 3 is a perspective exploded view of the coupling housing together with the working part; and

FIG. 4 is a perspective view of the working part inserted into the coupling housing.

### DESCRIPTION OF THE PREFERRED EMBODIMENTS

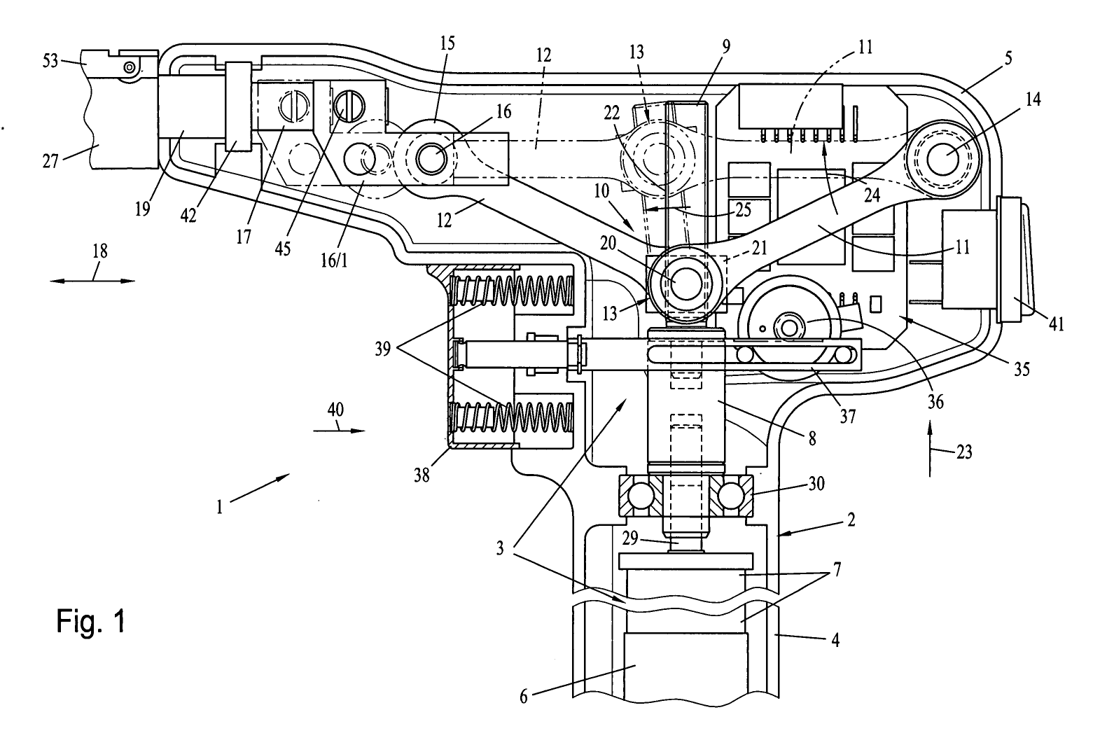

Referring to the drawings in particular, FIG. 1 shows a partial section of a surgical instrument 1 according to the present invention, which has a grip part 2, in which an actuating drive 3 is integrated. This actuating drive 3 comprises in this exemplary embodiment an electric motor 6, which is accommodated in the handle 4 of the housing 5 and is coupled with a push rod 9 via a gear mechanism 7 and a joint coupling 8. The housing 5 has a two-part design in this exemplary embodiment and comprises two “half shells,” of which only the rear one is visible in the drawing.

The push rod 9 is in functional connection with a lever mechanism 10, which is designed as a toggle lever. This lever mechanism 10 comprises a support lever 11 and a pushing lever 12, which are connected with one another in an articulated manner via an intermediate joint 13. At its end located opposite the intermediate joint 13, the support lever 11 is mounted rotatably in the grip part 2 or the housing 5 of the said grip part by means of a corresponding mounting pin 14.

At its end located opposite the intermediate joint 13, the pushing lever 12 has a bearing eye 15, with which the pushing lever 12 is connected to a prismatic joint 16/1 via a mounting pin 16 in an articulated manner. This prismatic joint 16/1 is in connection with a ram 17, and the said ram 17 is accommodated in a bearing bush 19 in an axially adjustable manner in the direction of the double arrow 18. This bearing bush 19 is arranged stationarily in the housing 5 of the grip part 2, as this is apparent from FIG. 1.

The push rod 9 is designed as a threaded spindle in this exemplary embodiment and can be driven rotatingly via the electric motor 6, the gear mechanism 7 as well as the joint coupling 8. A threaded block 21, which is in functional gear connection with the push rod 9 or the threaded section 22 of the said push rod, is provided between the support lever 11 and the pushing lever 12 in the area of the intermediate joint 13, which is provided with corresponding mounting pins 20 (of which only the front one is visible in the drawing) for the articulated connection of the support lever 11 and the pushing lever 12.

It is easy to imagine that during the rotation of the push rod 9, the threaded block 21 is moved in the axial direction of the push rod 9 from the starting position shown in FIG. 1 along the push rod 9 in the direction of arrow 23. Due to this adjusting movement of the threaded block 21 and consequently also the mounting pin 20, the support lever 11 is pivoted around its mounting pin 14 in the direction of arrow 24. The intermediate joint 13 with its mounting pin 20 moves on a circular path during this pivoting movement, so that the push rod 9 is deflected around the joint coupling 8 in the direction of arrow 25 and reaches the end position shown by phantom lines at the end of the movement. At the same time, the pushing lever 12 is pivoted around the prismatic joint 16, so that, on the whole, a translational motion of the ram 17 to the outside in the direction of the double arrow 18 is brought about by this toggle lever arrangement. At the end of the movement, the support lever 11 and the pushing lever 12 assume a “stretched,” at least approximately aligned position, as this is indicated by phantom lines in FIG. 1.

As is apparent from FIG. 4, this ram 17 is in positive-locking connection with a working part 26, and this working part 26 is provided at its free end with a tool part, which is not shown in the drawing. This working part 26 is inserted into a coupling housing 27, which is in turn fastened tightly to the bearing bush 19 projecting from the housing 5 axially in the outward direction.

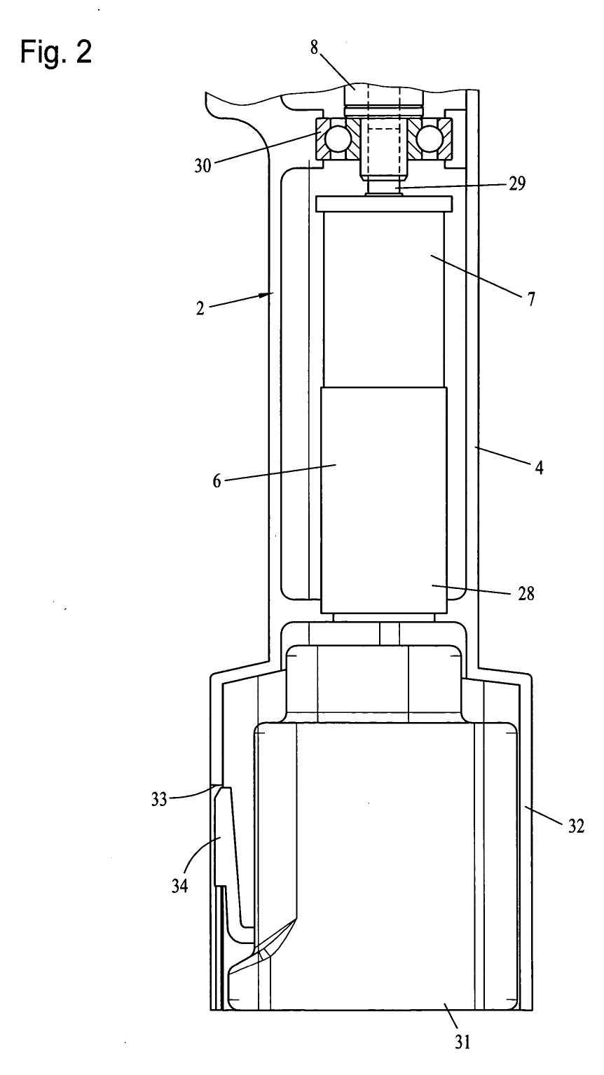

In this exemplary embodiment, the electric motor 6 is tightly inserted with its lower end 28 into the handle 4 of the housing 5, as is apparent from FIG. 2. A rolling bearing 30, which is stationarily received in the upper end area of the handle 4, is provided in the upper end area between the gear mechanism 7 and the joint coupling 8 for the rotatable mounting of the output shaft 29 of the gear mechanism 7.

The electric motor 6 is supplied with power in this exemplary embodiment by a power storage pack 31, which is formed, for example, by rechargeable batteries. This energy storage pack 31 is replaceably inserted into a receiving housing 32, and, as is apparent from FIG. 2, this receiving housing adjoins the lower end of the handle 4 in one piece. For securing in the receiving housing 32, this housing has an opening 33 on its side, with which a snap-in pin 34 of the power storage pack 31 can be engaged in a positive-locking manner.

An electronic control means 35 (FIG. 1), which is provided with a measuring means 36, for example, in the form of an incremental shaft encoder, is provided to control the movement process of the lever mechanism 10.

This incremental shaft encoder 36 is in gear connection with an actuating element 38 designed as a pushbutton via a toothed rack 37. The pushbutton 38 can be moved against the spring force of two resetting springs 39 in the direction of arrow 40, and this adjusting movement is transmitted to the toothed rack 37. This translational motion is transmitted via the teeth of the toothed rack 37, which are not shown specifically in FIG. 1, to the incremental shaft encoder 36, and the pulse signals generated as a result are evaluated by the control means 35.

These pulse signals of the shaft encoder 36 are converted by the control means 35 into control signals for actuating the electric motor 6, which is designed as a motor operator. Provisions are made in this connection for the motor operator 6 to be able to be actuated by the control means 35 such that the translational pushing motion or working movement in the direction of the double arrow 18 of the ram 17 is proportional to the adjustment path of the pushbutton 38.

Due to this proportional control of the motor operator 6, which is provided in this exemplary embodiment, a tool part arranged at the outer end of the working part 26 can be controlled in a controlled manner and this tool part performs a controlled working movement.

Both the control means 35 and the motor operator 6 can be switched off and on via a main switch 41 provided in the rear end area of the housing 5.

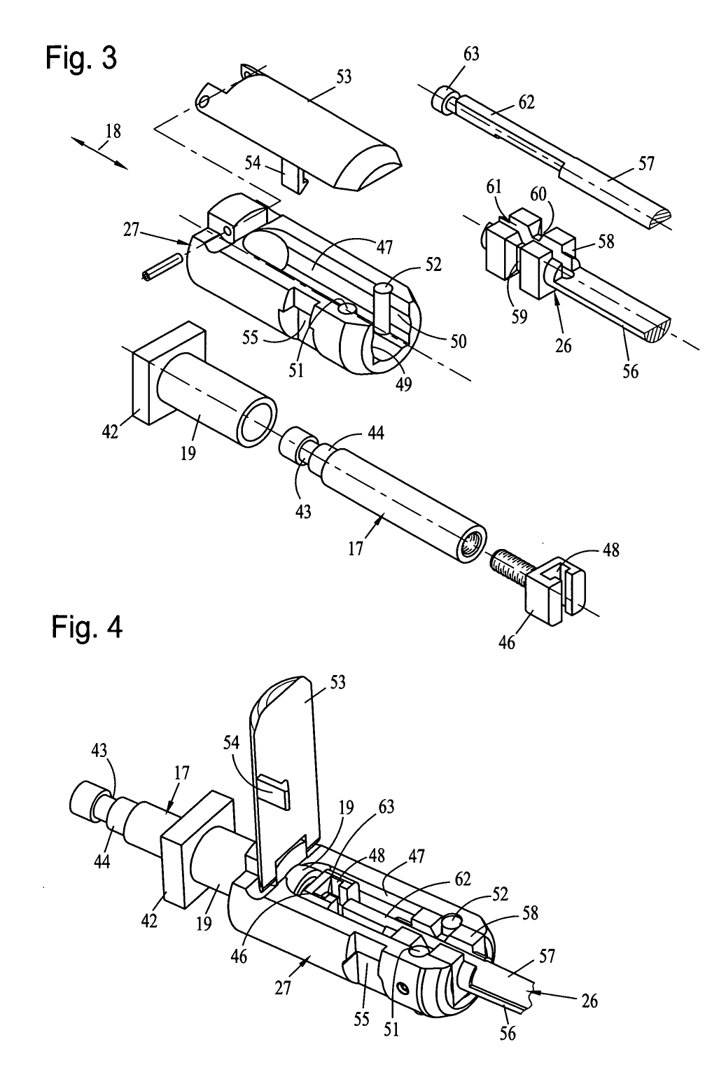

The coupling housing 27 is provided to make it possible to use the actuating drive 3 arranged in the grip part 2 with different surgical tool parts or working parts. This coupling housing 27 is used to replaceably receive different working parts 26, as this is apparent as an example from the perspective exploded view in FIG. 3.

It can be recognized from FIG. 3 that in its end area located opposite the attached coupling housing 27, the bearing bush 19 has a bearing flange 42, which is designed as a square plate, and with which the bearing bush 19 can be tightly inserted into the housing 5 of the grip part 2, as this is shown as an example in FIG. 1. Furthermore, it can be recognized from FIG. 3 that the ram 17 has a cylindrical design and is plugged into the bearing bush 19. At its end protruding inwardly from the bearing bush 19, the ram 17 has multiple steps and has a mounting groove 43, which is part of a radially tapered mounting pin 44.

The prismatic joint 16/1 can be attached to this mounting pin 44 and can be mounted tightly thereon by means of a securing screw 45 (FIG. 1). At its end located opposite the mounting pin 44, the ram 17 has a mounting block 46, which protrudes into a receiving chamber 47 of the coupling housing 27 in the mounted state shown in FIG. 4. This mounting block 46 is provided with a double-T groove 48, which is open on the front side and whose function will be explained in greater detail below. Furthermore, it can be recognized from FIG. 3 that the mounting block 46 can be screwed together with the ram 17. It is possible due to this design to adapt the ram 17 to coupling elements of different designs of a working part in a simple manner by replacing the mounting block 46.

As is also apparent from FIG. 3, the mounting groove 47 has a mounting pin 51 and 52 each in its end area located opposite the bearing bush 19 in the area of its two side walls 49 and 50. These two mounting pins 51 and 52 are arranged opposite each other and protrude by about half their diameter into the receiving chamber 47 of the coupling housing 27.

Furthermore, the coupling housing 27 is provided with a housing cover 53, which is mounted on the coupling housing 27 in an articulated manner in the area of the bearing bush 19. This housing cover 53 is provided in the area of one of its longitudinal edges with a locking finger 54, which can be caused to engage a locking groove 55 of the coupling housing 27 in a positive-locking manner in the closed state.

Furthermore, it can be seen from FIG. 3 that the working part 26 has a kind of guide shaft 56, along which a slide 57 is mounted adjustably. In its end area located toward the coupling housing 27, the guide shaft 56 is provided with a mounting block 58, which is provided with two lateral mounting grooves 59 and 60. The guide shaft 56 with its mounting block 58 can be caused to tightly engage the two mounting pins 51 and 52 with these mounting grooves 59 and 60 when the mounting block 58 is inserted into the receiving chamber 47 of the coupling housing 27 in the area of these mounting pins 51 and 52.

As is also apparent from FIG. 3, the mounting block 58 has an upwardly open receiving groove 61, into which the slide 57 can be inserted. The slide 57 is provided for this purpose in its end area recognizable in FIG. 3 with a web-like bearing section 62, with which the slide 57 can be inserted into the receiving groove 61 of the guide shaft 56 fittingly, axially adjustably and nontwistably. At its free end protruding from the mounting block 58 of the guide shaft 56, the bearing section 62 of the slide 57 is provided with a radially expanded head part 63, with which the bearing section 62 can be caused to engage the double-T groove 48 of the ram 17 in a positive-locking manner when the guide shaft 56 is inserted together with the slide 57 into the receiving chamber 47.

Due to this embodiment of the guide shaft 56 with its mounting block 58, which embodiment is shown as an example, and due to the design of the slide 57 with its head part 63, this working part 26 can thus also be inserted into the receiving chamber 47 of the coupling housing 27 in a very simple manner.

Due to the positive-locking connection between the head part 63 and the receiving block 46 of the slide 17, the axial pushing movement in the direction of the double arrow 18 of the ram 17 can be transmitted to the slide 57 with an extremely small clearance. This working movement is mechanically transmitted, furthermore, to a tool part, which is arranged correspondingly at the other end of the working part 26 and which is not shown in the drawings, because numerous forms of these designs are already known from the state of the art, as was indicated, for example, in the introduction to the specification.

FIG. 4 shows the working part 26 in the mounted state. It can be recognized that the mounting block 58 with its two mounting grooves engages the two mounting pins 51 and 52 of the coupling housing 27 in a positive-locking manner. Due to the corresponding axial setting of the slide 57 in relation to the guide shaft 56, this will also become connected by its head part 63 with the double-T groove 48 of the receiving block 46 in a positive-locking manner. After the housing cover 53 is closed and the locking finger 54 snaps into the locking groove 55, the working part 26 is thus tightly secured in the receiving chamber 47 of the coupling housing 27. The tight mounting of the coupling housing 27 in the bearing bush 19 can be achieved, for example, by means of clamping screws, which, not visible in the drawing, are screwed into the coupling housing 27, e.g., from below and press the jacket surface of the bearing bush 19 seated in the coupling housing 27, so that a sufficient clamping action is generated.

Instead of the actuating drive in the form of the electric motor with its gear mechanism, which is described as an example, it is also possible to use a pneumatic cylinder with a corresponding servo valve control. The cylinder is inserted in this case into the housing 5 of the grip part 2 in the area of the electric motor 6 and is connected by its piston rod with the lever mechanism 10. The use of a pneumatic cylinder permits two basic functional positions, namely, the starting position indicated by solid lines in FIG. 1 as well as the end position indicated by phantom lines in FIG. 1, in which the two levers 11 and 12 are at least approximately aligned. This embodiment with the pneumatic cylinder as the actuating drive is not explicitly shown in the drawings.

However, it is easy to imagine that the piston rod of the pneumatic cylinder is coupled by its free end with the intermediate joint 13 of the lever mechanism 10 in an articulated manner. To make possible the pivoting movement during the working stroke of the piston rod in the direction of arrow 25, the pneumatic cylinder is mounted pivotably in the handle 4 of the housing 5. Furthermore, the receiving housing 32 is not provided in this embodiment, because the power supply or the pressure supply for the pneumatic cylinder is performed by means of an external compressor and a corresponding delivery connection. The actuating element 38 can now switch the servo valve for actuating the pneumatic cylinder mechanically or electrically. The servo valve is the control means in both cases, which is in turn actuated by the actuating element 38 and correspondingly admits the necessary system pressure to the pneumatic cylinder to move it in one direction or the other.

While specific embodiments of the invention have been shown and described in detail to illustrate the application of the principles of the invention, it will be understood that the invention may be embodied otherwise without departing from such principles.

1. A surgical instrument, comprising:
a grip part;
a working part having a tool part in an end area provided with a slide, which is adjustable in relation to said working part and has an adjusting movement that brings about a working movement of said tool part;
actuating element which can be actuated manually;
an actuating drive for bringing about said adjusting movement, said actuating drive being actuated via said actuating element, said actuating drive including a lever mechanism;
a control means controlled via said actuating element for actuating said actuating drive, said control element being arranged in the area of said grip part, said actuating drive bringing about the working movement of said slide depending on the position of said actuating element and being mechanically coupled with said slide via said lever mechanism.

2. A surgical instrument in accordance with claim 1, wherein
said lever mechanism has a support lever and a pushing lever;
said support lever and said pushing lever are connected rotatably with one another via an intermediate joint;
said support lever is mounted rotatably with an end located opposite said intermediate joint in said grip part and said pushing lever is connected by its end located opposite said intermediate joint with said slide in an articulated manner via a prismatic join;
said support lever and said pushing lever can be brought by said actuating drive from an angular position into an at least approximately aligned position for driving said slide.

3. A surgical instrument in accordance with claim 1, wherein said actuating drive has an electric motor, by which a push rod mechanically coupled with said lever mechanism can be driven.

4. A surgical instrument in accordance with claim 3, wherein said electric motor is in gear connection with said push rod via a planet gear.

5. A surgical instrument in accordance with claim 3, wherein said electric motor can be actuated via said control means such that said tool part can be adjusted from a starting position into an end position and vice versa.

6. A surgical instrument in accordance with claim 4, wherein:
said electric motor is designed as a motor operator;
an adjustment path of said actuating element can be detected by means of a measuring means and transmitted to said control means; and
said motor operator can be actuated by said control means such that the working movement of said slide of said working part is essentially proportional to the adjustment path of said actuating element.

7. A surgical instrument in accordance with claim 6, wherein said measuring means is formed by a incremental transducer with an incremental shaft encoder.

8. A surgical instrument in accordance with claim 1, wherein said actuating drive is formed by a double-acting or single-acting pneumatic cylinder arranged integrated in said grip part, and that a pneumatic control valve, which can be actuated via said actuating element, is provided as the control means for controlling the adjusting movement of said pneumatic cylinder.

9. A surgical instrument in accordance with claim 1, wherein said working part is detachably connected by its said tool part with said grip part.

10. A surgical instrument in accordance with claim 2, wherein said grip part has a housing, which is provided with a coupling housing for coupling with said working part.

11. A surgical instrument in accordance with claim 10, wherein:
said coupling housing has a closable receiving chamber, into which said working part with a guide shaft can be replaceably inserted;
said slide axially protrudes from said guide shaft into said receiving chamber;
said prismatic joint is provided with a ram, which protrudes into said receiving chamber and with which said slide can be engaged in a positive-locking and detachable manner.

12. A surgical instrument in accordance with claim 11, wherein said receiving chamber of said coupling housing can be closed with a housing cover, which is pivotably mounted on said coupling housing, and that said working part is held by said closed housing cover in said receiving chamber.

13. A surgical instrument in accordance with claim 8, wherein different working parts with said different tool parts are provided for different intended uses, which said tool parts can be replaceably mounted on said grip part.
